(19) United States
(12) Patent Application Publication
(10) Pub. No.: US 2009/0139889 A1
(43) Pub. Date: Jun. 4, 2009

(54) Medical Instrument Sterilization Pouch

(76) Inventor: George Kyritsis, Laval (CA)

Correspondence Address: NEUSTEL LAW OFFICES, LTD., 2534 SOUTH UNIVERSITY DRIVE, SUITE 4, FARGO, ND, 58103, US

(21) Appl. No.: 12/366,416

(22) Filed: Feb. 5, 2009

Related U.S. Application Data
(63) Continuation-in-part of application No. 11/622,942, filed on Jan. 12, 2007.

(51) Int. Cl. A61B 19/02

(52) U.S. Cl. 206/439

(57) ABSTRACT

A medical instrument sterilization pouch for efficiently bagging hinged instruments. The medical instrument sterilization pouch generally includes a first layer comprised of a gas permeable material, a second layer comprised of a gas impermeable or permeable material and a sealing edge substantially extending along an outer perimeter of the first layer and the second layer to attach the first layer to the second layer. The sealing edge defines a sealed inner space between the first layer and the second layer for receiving a medical instrument, wherein the sealing edge includes an inner portion extending inwardly from an outer end of the sealing portion to define a barrier between a first lower portion and a second lower portion of the pouch within the inner space.

### CROSS REFERENCE TO RELATED APPLICATIONS

I hereby claim benefit under Title 35, United States Code, Section 120 of U.S. patent application Ser. No. 11/622,942 filed Jan. 12, 2007. This application is a continuation-in-part of the Ser. No. 11/622,942 application. The Ser. No. 11/622,942 application is currently pending. The Ser. No. 11/622,942 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to sterilization pouches and more specifically it relates to a medical instrument sterilization pouch for efficiently bagging hinged instruments.

2. Description of the Related Art

Any discussion of the prior art throughout the specification should in no way be considered as an admission that such prior art is widely known or forms part of common general knowledge in the field.

Sterilization pouches have been in use for years. Typically, when in a medical, dental or other similar environment it is necessary to keep all the instruments in pouches prior to use. The pouches effectively serve to prevent germs or other harmful toxins from coming in contact with the instruments when the instruments are not being used. The instruments are also generally sterilized prior to being inserted into the pouch or sterilized while inserted within the pouch.

Sterilization pouches are manufactured from many different materials, sizes and with many different sealing mechanisms. Although, one universal feature that generally exists in all sterilization pouches is that the sterilization pouches are generally rectangular shaped. This poses a problem with respect to hinged instruments, such as but not limited to orthodontic pliers and surgical pliers in that the hinged instruments can not adequately fit on their respective tool racks (i.e. pliers rack) once the hinged instruments are in the pouch. In addition, the prior art does not allow hinged instruments to be sterilized in an open or unlocked position. Because of the general lack of efficiency and practicality in the prior art there is the need for a new and improved medical instrument sterilization pouch for efficiently bagging hinged instruments.

### BRIEF SUMMARY OF THE INVENTION

The general purpose of the present invention is to provide a medical instrument sterilization pouch that has many of the advantages of the sterilization pouches mentioned heretofore. The invention generally relates to a sterilization pouch which includes a first layer comprised of a gas permeable material, a second layer comprised of a gas permeable or impermeable material and a sealing edge substantially extending along an outer perimeter of the first layer and the second layer to attach the first layer to the second layer. The sealing edge defines a sealed inner space between the first layer and the second layer for receiving a medical instrument, wherein the sealing edge includes an inner portion extending inwardly from an outer end of the sealing portion to define a barrier between a first lower portion and a second lower portion of the pouch within the inner space.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

An object is to provide a medical instrument sterilization pouch for efficiently bagging hinged instruments.

Another object is to provide a medical instrument sterilization pouch that accommodates a variety of different style hinged instruments.

An additional object is to provide a medical instrument sterilization pouch that may be utilized with non-hinged instruments.

A further object is to provide a medical instrument sterilization pouch that allows hinged instruments to be sterilized in an open position.

Another object is to provide a medical instrument sterilization pouch that allows the hinged instruments to be placed on their respective holding racks (i.e. pliers rack) after the hinged instruments are bagged.

Another object is to provide a medical instrument sterilization pouch that includes a durable outer material to prevent puncture by the instrument.

Another object is to provide a medical instrument sterilization pouch that mimics the shape and size of the medical instruments utilized thus producing less environmental and biological waste.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention in use and positioned upon a tool rack.

FIG. 2 is a front view of the present invention.

FIG. 3 is an upper perspective view of the present invention with a medical instrument partially inserted within the pouch.

FIG. 4 is an upper perspective view of the present invention with a medical instrument inserted within the pouch.

FIG. 5 is an upper perspective view of the present invention with a medical instrument inserted within the pouch and the flap sealed upon the pouch.

FIG. 6 is a sectional view taken along lines 6-6 of FIG. 2.

FIG. 7 is a front view of a pouch illustrating a first step in a first example of manufacturing the present invention.

FIG. 8 is a front view of a pouch illustrating a second step in the first example of manufacturing the present invention.

FIG. 9 is a front view of a pouch illustrating a first step in a second example of manufacturing the present invention.

FIG. 10 is a front view of a pouch illustrating a second step in the second example of manufacturing the present invention.

FIG. 11 is a front view of a first alternative embodiment of the present invention.

FIG. 12 is a front view of a second alternative embodiment of the present invention.

FIG. 13 is a front view of a third alternative embodiment of the present invention.

FIG. 14 is a front view of a fourth alternative embodiment of the present invention.

FIG. 15 is a front view of a fifth alternative embodiment of the present invention.

FIG. 16 is a front view of a sixth alternative embodiment of the present invention.

FIG. 17 is a front view of a seventh alternative embodiment of the present invention.

FIG. 18 is an upper perspective view of the new embodiment of the present invention illustrating the inner portion being completely sealed and extending within the inner space of the pouch to separate the legs of the hinged instrument.

FIG. 19 is a top view of the new embodiment.

FIG. 20 is a sectional view taken along lines 20-20 of FIG. 19.

FIG. 21 is a top view of the new embodiment illustrating cutting lines or perforations extending through the inner portion to form a recessed portion.

FIG. 22 is a top view of an alternate embodiment of the pouch illustrating a differing in shape inner portion.

FIG. 23 is a top view of the alternate embodiment of FIG. 22 with a cutout extending through a part of the inner portion.

FIG. 24 is a top view of another alternate embodiment of the pouch illustrating the inner portion separated from the rest of the sealing edge.

FIG. 25 is a top view of yet another alternate embodiment of the pouch illustrating the inner portion comprised of an ovular shaped configuration.

FIG. 26 is a top view of the alternate embodiment of FIG. 25 with a cutout extending through the inner portion.

### DETAILED DESCRIPTION OF THE INVENTION

### A. Overview

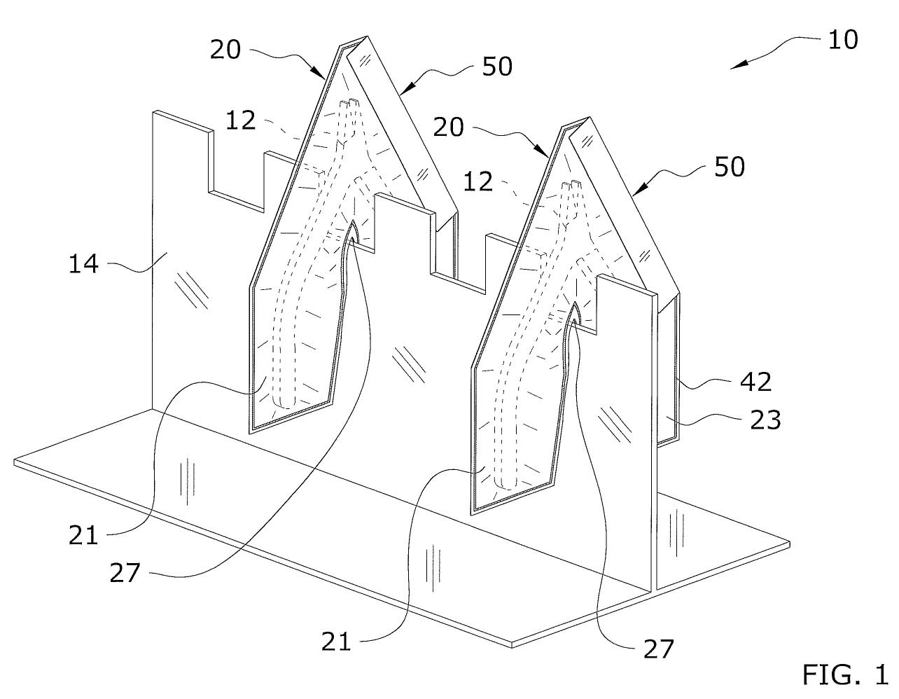

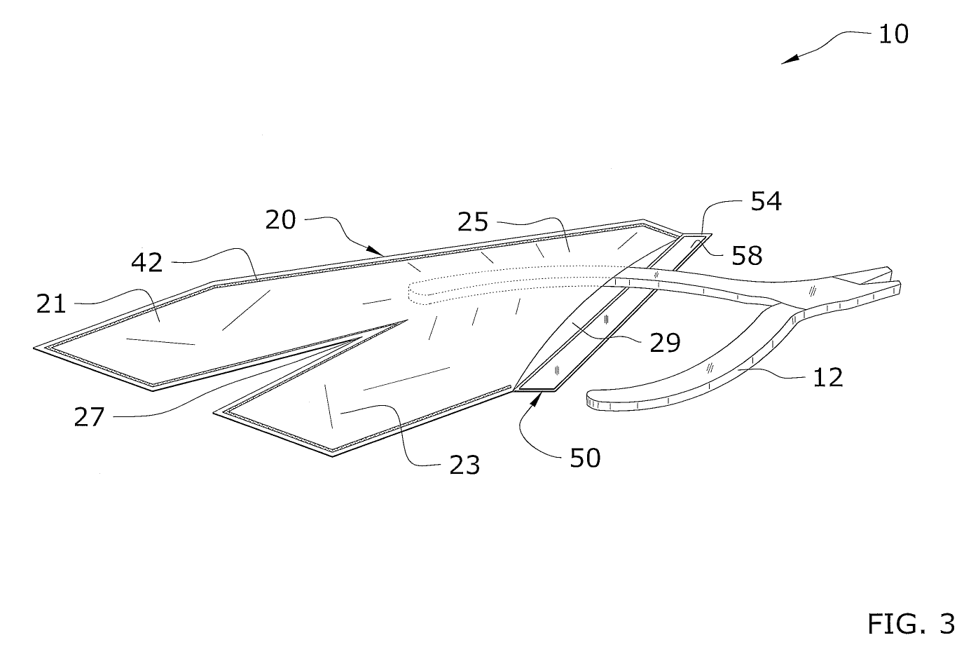

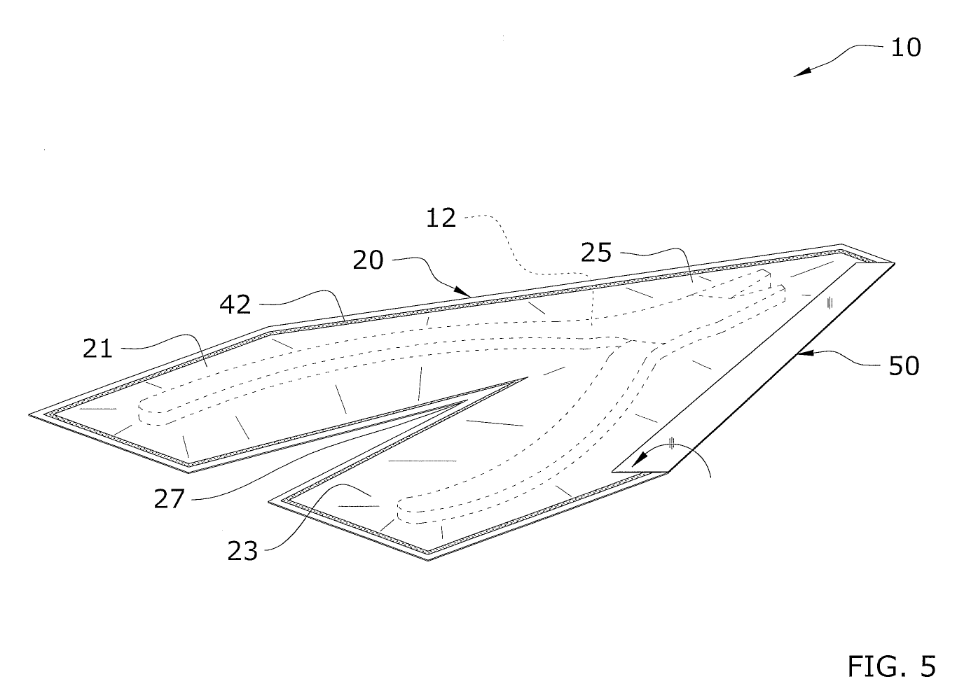

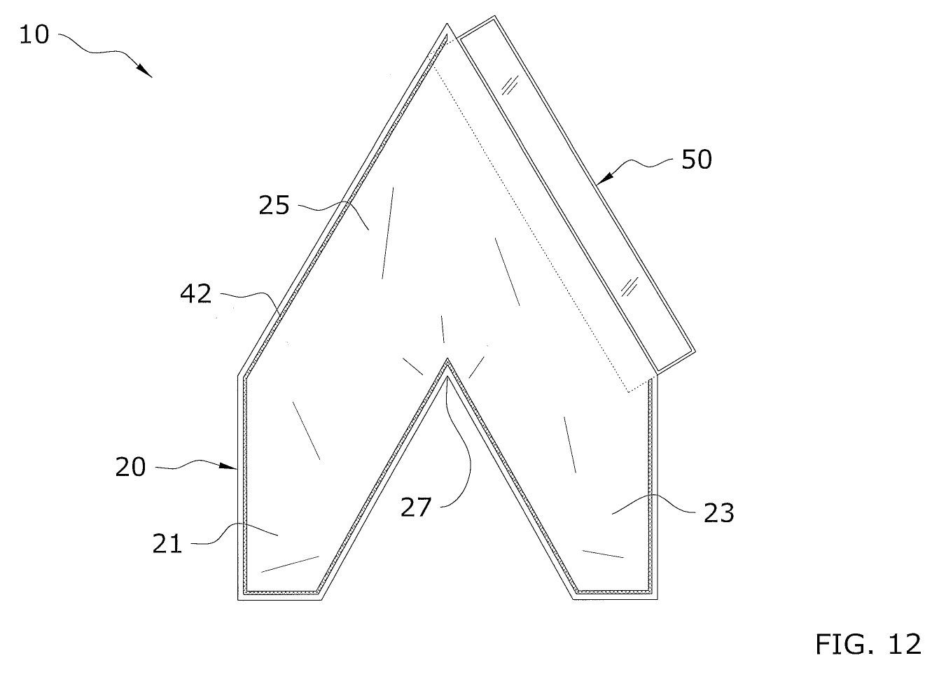

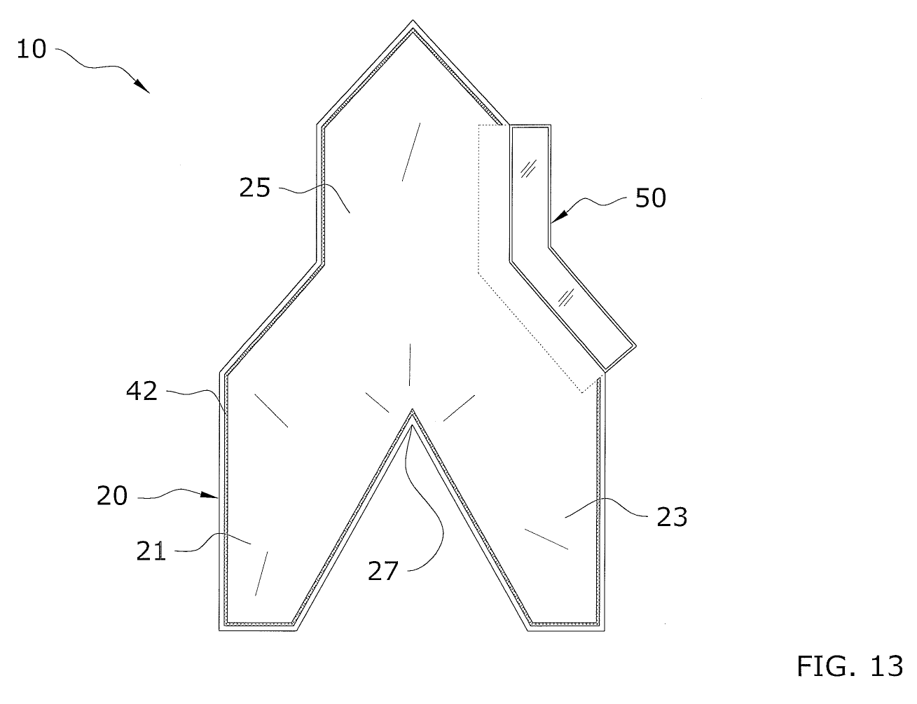

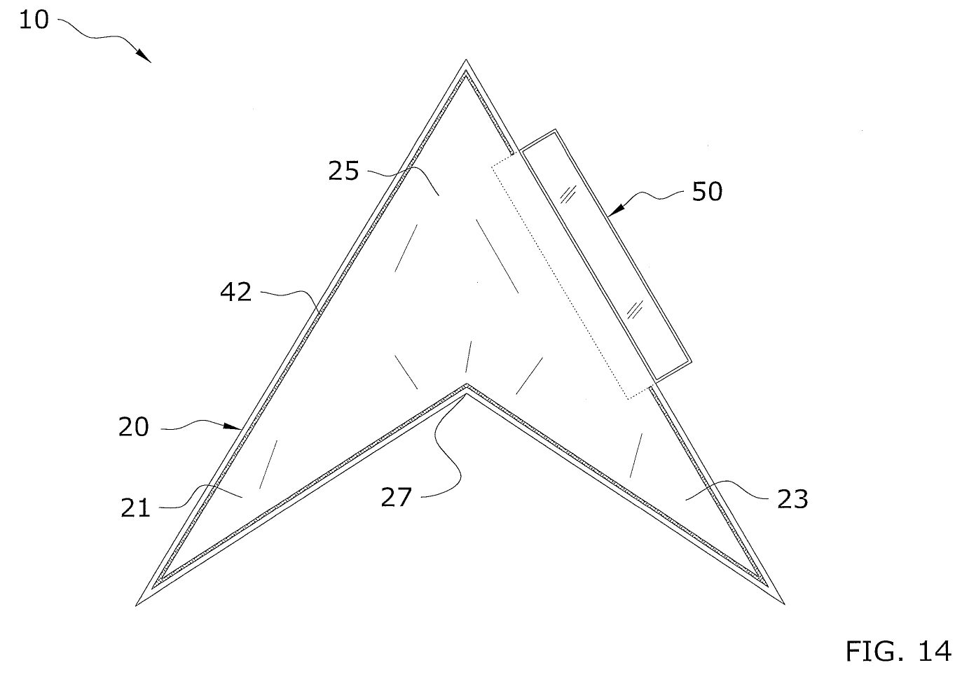

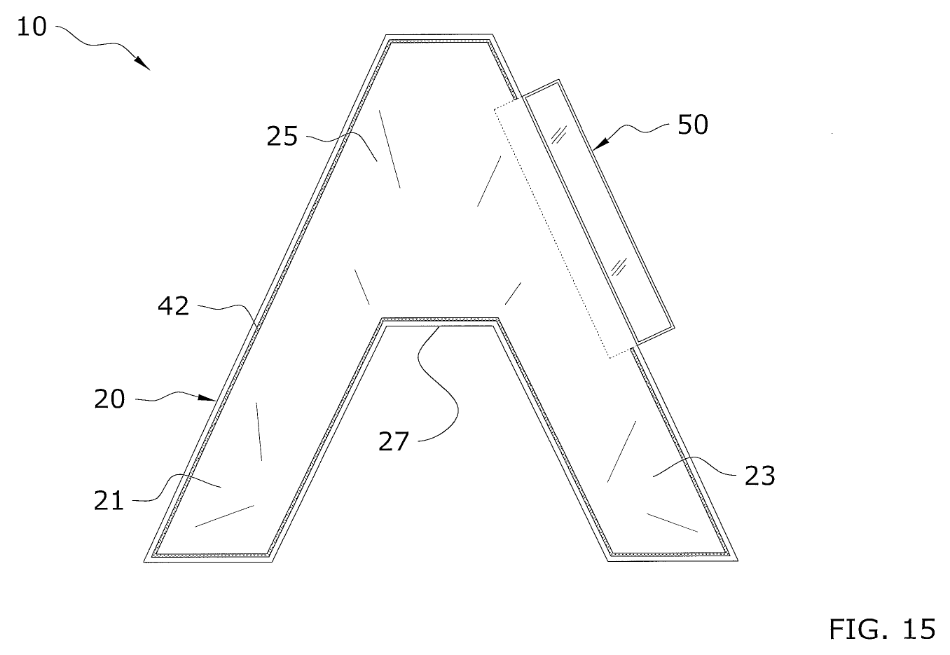

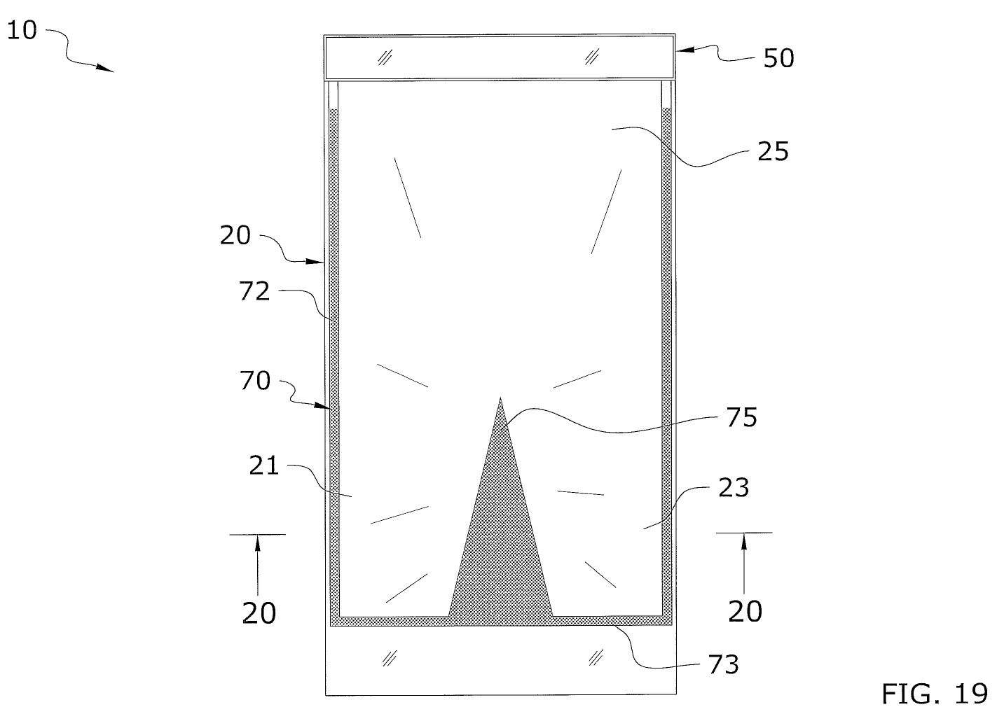

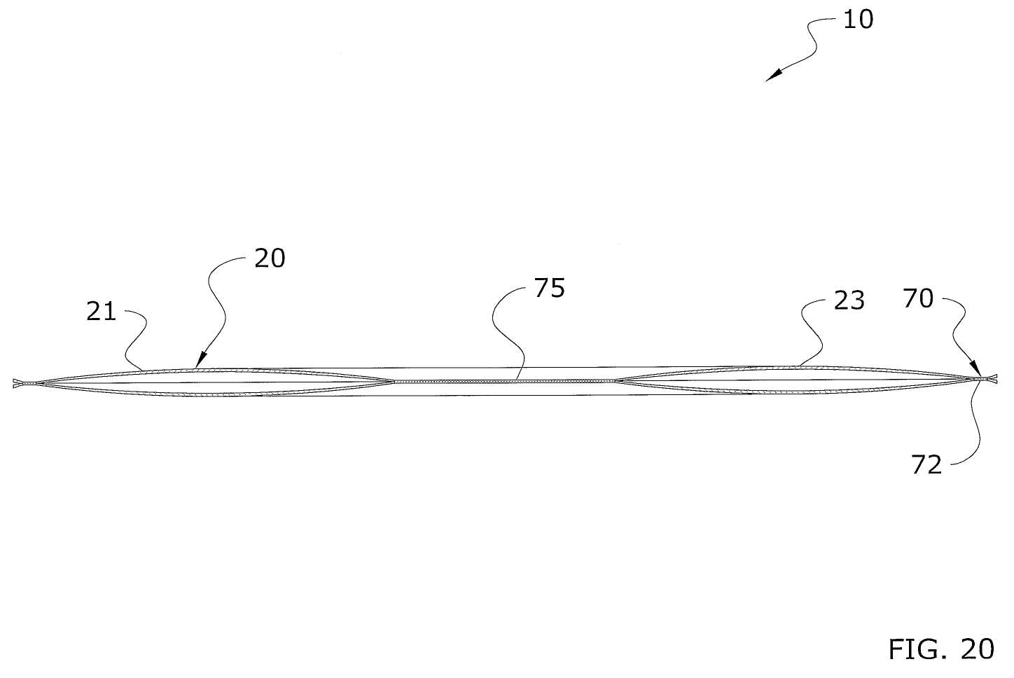

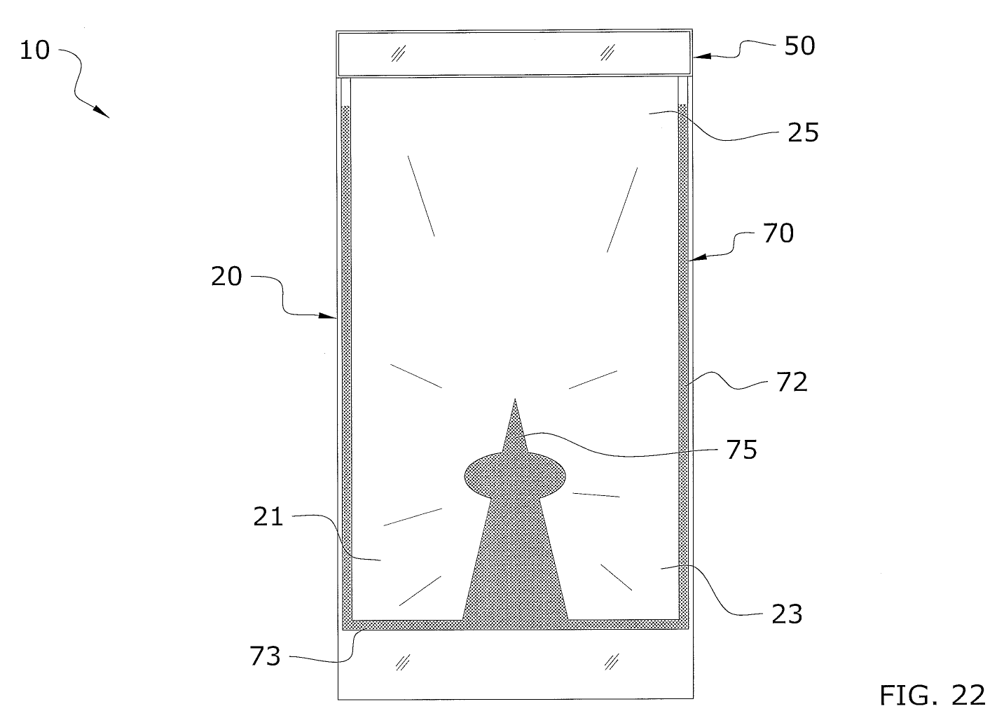

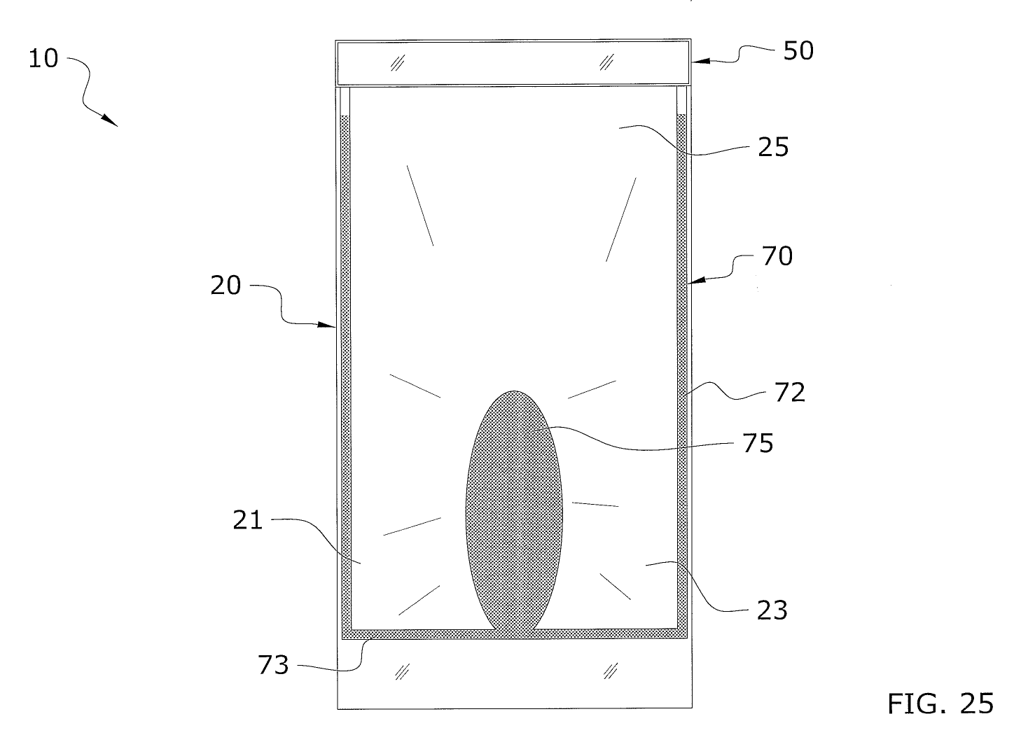

Turning now descriptively to the drawings, in which similar reference characters denote similar elements throughout the several views, FIGS. 1 through 26 illustrate a medical instrument sterilization pouch 10, which comprises a pouch 20 including a first layer 30 and a second layer 40, wherein the first layer 30 and/or the second layer 40 are comprised of a gas permeable material and wherein the first layer 30 is attached to the second layer 40 via an outer seal 42 substantially surrounding an outer perimeter of the first layer 30 and the second layer 40. The pouch 20 includes a first lower portion 21 and a second lower portion 23, wherein the first lower portion 21 is separated from the second lower portion 23 and wherein the first lower portion 21 and the second lower portion 23 form a recessed portion 27 between thereof. The pouch 20 preferably receives a medical instrument 12 (i.e. surgical pliers), wherein the medical instrument 12 is sterilized within the pouch 20 and is then positioned upon a respective tool rack 14 (i.e. pliers rack).

### B. Pouch

The pouch 20 is preferably comprised of a configuration to hold hinged medical instruments 12, such as but not limited to orthodontic pliers, surgical pliers, tweezers and scissors. The pouch 20 also preferably adequately holds non-hinged medical instruments 12, such as but not limited to inspection mirrors, dental picks, dental scalers and spatulas, wherein the non-hinged medical instruments 12 are simply inserted into the pouch 20 in a crossed manner. It is also appreciated that the pouch 20 may hold various other tools or equipment not associated with medical use. The pouch 20 preferably mimics the shape of the medical instrument 12, thus requiring the minimal amount of packaging for each medical instrument 12.

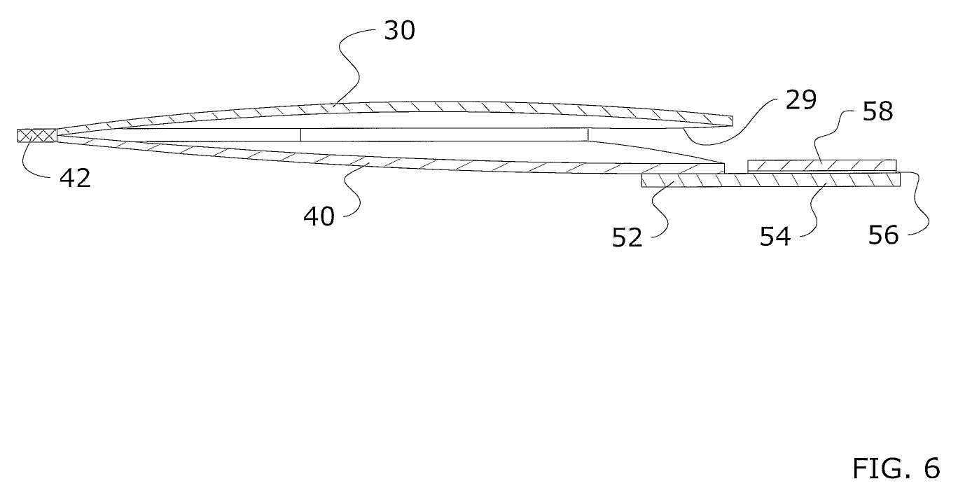

The pouch 20 includes a first layer 30 and a second layer 40 as shown in FIG. 6. The first layer 30 and the second layer 40 are preferably both comprised of a gas permeable material. The first layer 30 and the second layer 40 are further impermeable to micro-organisms or toxins. The first layer 30 and the second layer 40 are further preferably comprised of a material resistant enough to withstand both steam and dry heat sterilization cycles. The first layer 30 and the second layer 40 may further be comprised of various materials, such as but not limited to nylon films, polypropylene films, polyethylene films, polyester polypropylene pellicles, blends of medical grade paper or a combination of two or more materials. The first layer 30 and the second layer 40 are further preferably comprised of a transparent material so as to easily view the medical instrument 12 within the pouch 20. The first layer 30 and the second layer 40 may also include chemical, steam and heat activated indicators. The indicators are preferably painted on the first layer 30 or the second layer 40.

The first layer 30 and the second layer 40 may be comprised of a plurality of different shapes and sizes, wherein each of the configurations of the first layer 30 and the second layer 40 forms a recessed portion 27 to receive the legs of a medical instrument 12 as illustrated in FIGS. 1 through 17. The first layer 30 and the second layer 40 are further preferably comprised of substantially similar configurations.

The first layer 30 and the second layer 40 are preferably attached substantially near an outer perimeter of the first layer 30 and the second layer 40 via an outer seal 42 as shown in FIGS. 1 through 5. The outer seal 42 extends substantially along an entire perimeter of the first layer 30 and the second layer 40, wherein the outer seal 42 leaves room for an opening 29 along at least one outer edge to access the interior between the first layer 30 and the second layer 40.

The pouch 20 includes a first lower portion 21, a second lower portion 23 and an upper portion 25 opposite the first lower portion 21 and the second lower portion 23. The first lower portion 21 and the second lower portion 23 are preferably separably formed, wherein each lower portion preferably receives a respective leg of a medical instrument 12. The recessed portion 27 is formed between the first lower portion 21 and the second lower portion 23 as shown in FIGS. 1 through 5. The recessed portion 27 is preferably positioned upon a respective tool rack 14 (i.e. pliers rack) when storing or sterilizing the medical instrument 12 (i.e. surgical pliers) within the pouch 20 as illustrated in FIG. 1. Sterilizing the surgical pliers or other hinged instruments 12 upon the pliers rack 14 prevents the pouches 20 from being stacked upon one another, wherein stacking multiple medical instruments 12 upon one another may prevent the sterilization substance from coming in contact with the entire medical instrument 12. Utilizing the tool rack 14 allows the pouches 20 to be spaced adequately apart and thus allows the sterilization substance to effectively contact all surfaces of the medical instrument 12.

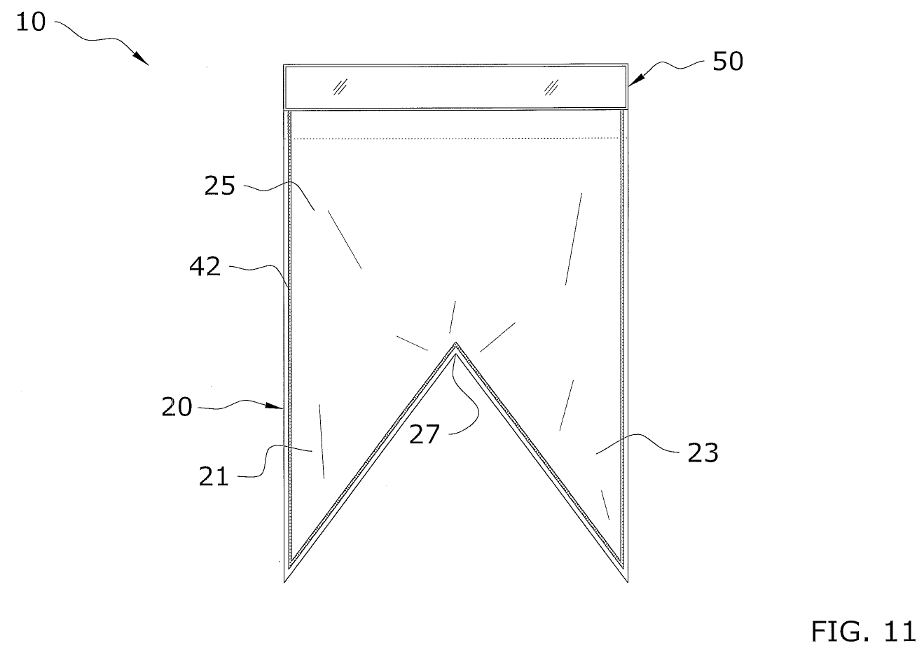

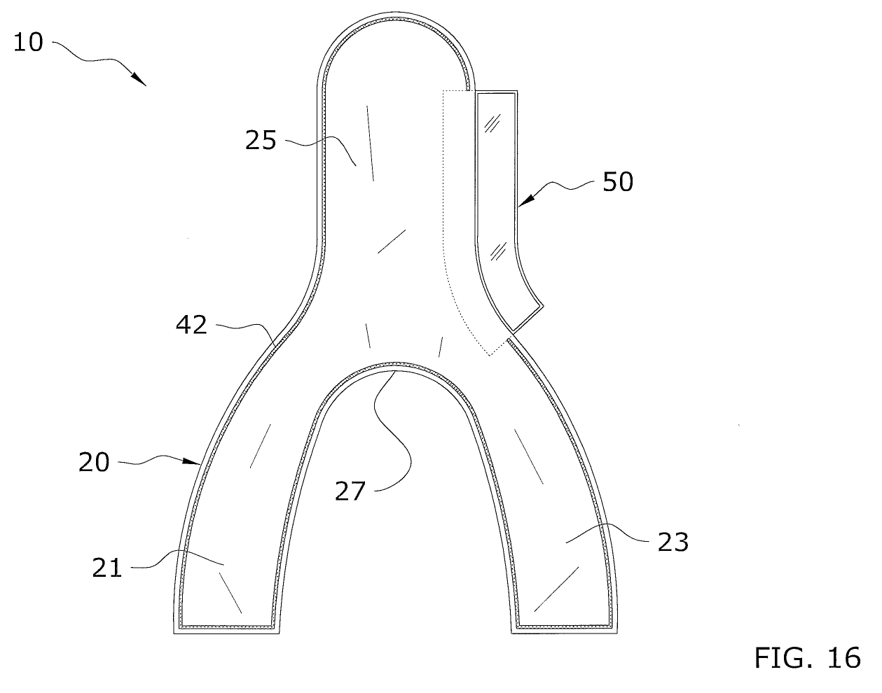

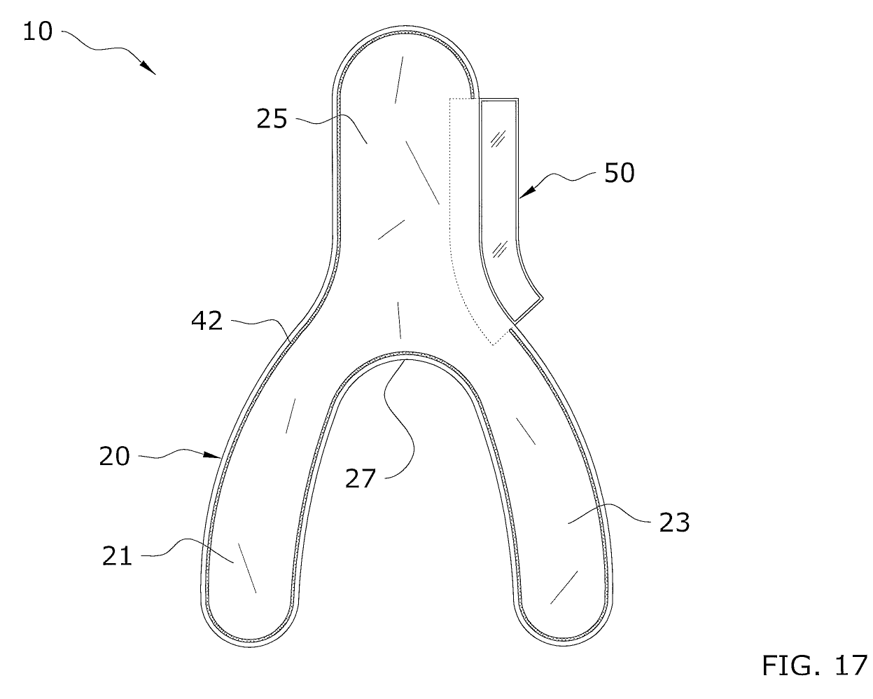

The recessed portion 27 is preferably comprised of a triangular shaped configuration. An apex of the recessed portion 27 preferably extends upwardly toward the upper portion 25 as shown in FIGS. 1 through 5. It is appreciated that the recessed portion 27 may also be comprised of a substantially semi-circular or oval shaped configuration as illustrated in FIGS. 16 and 17. It is further appreciated that the recessed portion 27 may be comprised of a plurality of various configurations all which substantially separate the first lower portion 21 from the second lower portion 23 as illustrated in FIGS. 11 through 15.

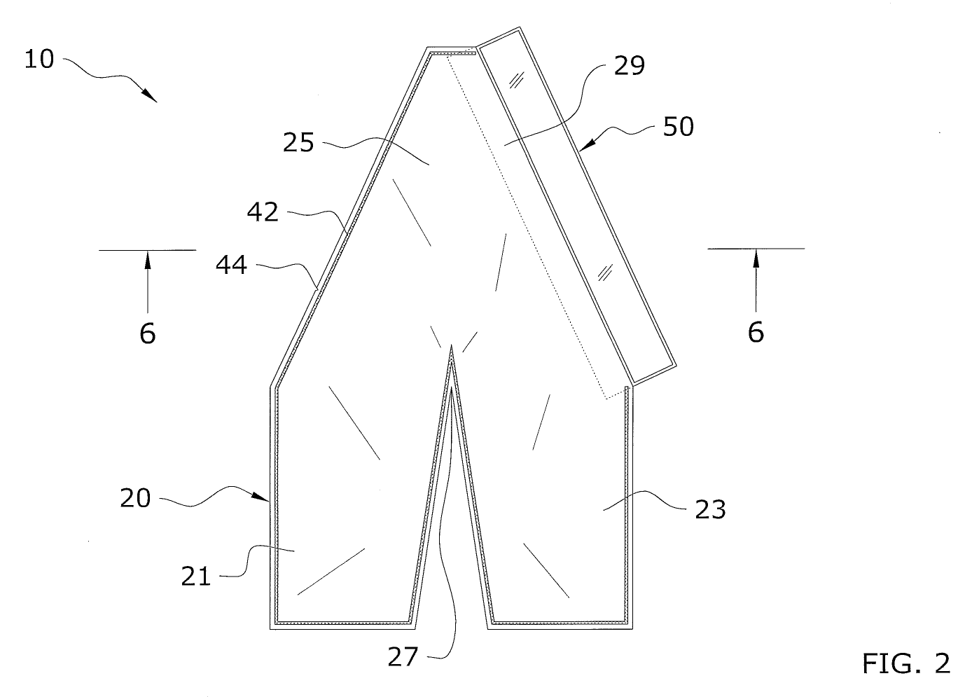

The first lower portion 21 and the second lower portion 23 preferably substantially mirror each other as shown in FIG. 2. The ends of the first lower portion 21 and the second lower portion 23 may also be comprised of a plurality of configurations, such as but not limited to flat, inclined or rounded as illustrated in FIGS. 2 and 11 through 17.

The upper portion 25 extends upwardly from lower portions 21, 23 and preferably receives the head of the medical instruments 12 as illustrated in FIGS. 1, 4 and 5. The upper portion 25 may also be comprised of a plurality of configurations, as illustrated in FIGS. 11 through 17. The upper portion 25 may include at least one tapered edge or a bottle neck configuration to better conform to the overall shape of the medical instrument 12 as illustrated in FIG. 2. The inside of the pouch 20 is preferably fluidly connected from the first lower portion 21 to the upper portion 25 to the second lower portion 23. The inside of the first lower portion 21 is preferably substantially fluidly sealed from the inside of the second lower portion 23 via the recessed portion 27.

The opening 29 is preferably formed along at least one edge of the upper portion 25 of the pouch 20 as shown in FIGS. 1 through 5, wherein the outer seal 42 does not extend across the perimeter of the first layer 30 and the second layer 40 along the opening 29. The opening 29 is further preferably positioned along a tapered end of the upper portion 25 as illustrated in FIGS. 1 through 5. The opening 29 allows access to the inside of the pouch 20 between the first layer 30 and the second layer 40. The opening 29 is also preferably large enough to allow the hinged instrument to be inserted adequately into the pouch 20.

### C. Flap

The flap 50 covers the opening 29 after the medical instrument 12 has been inserted within the pouch 20 to prevent contaminants and toxins from coming into contact with the medical instrument 12. The flap 50 preferably extends along an entire length of the opening 29 as shown in FIGS. 1 through 5. The flap 50 is further preferably parallel with the opening 29.

The flap 50 includes a first flange portion 52 and a second flange portion 54. The first flange portion 52 is preferably attached to the upper portion 25 and is substantially parallel with the opening 29. The second flange portion 54 preferably extends outwardly from the first flange portion 52 as shown in FIG. 6. The second flange portion 54 further preferably extends outwardly from the upper portion 25. The second flange portion 54 is also preferably parallel with the opening 29 as illustrated in FIG. 6.

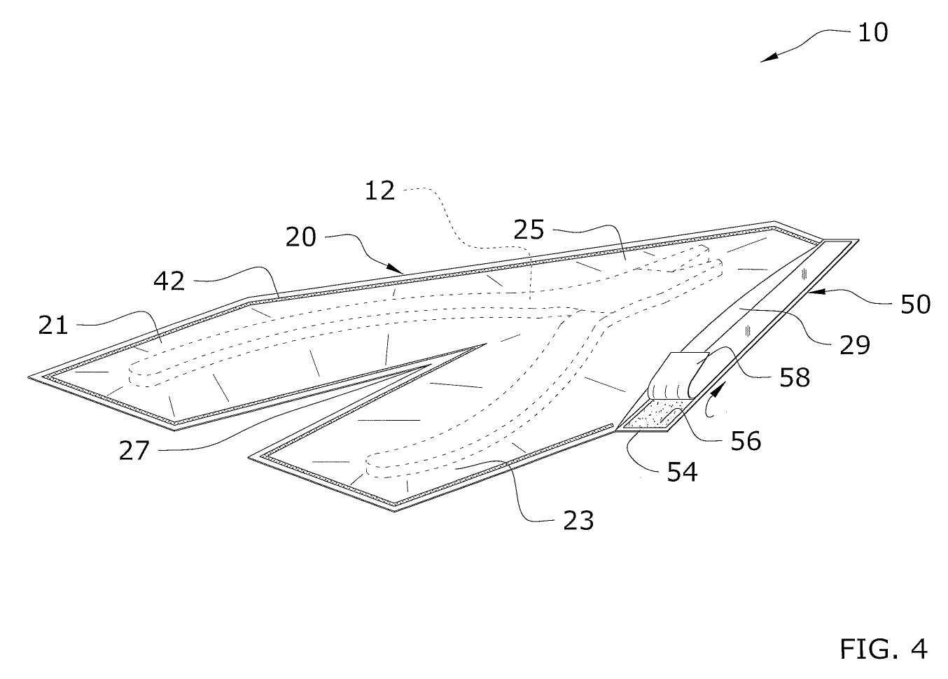

The second flange portion 54 preferably includes a sealing member 56 extending across a longitudinal axis of the second flange portion 54 as shown in FIG. 4. The sealing member 56 is positioned about an upper side of the second flange portion 54 of the flap 50. The sealing member 56 is further preferably comprised of an adhesive material so as to adequately stick to the upper side of the first layer 30. An outer layer 58 is preferably removably attached to the upper side of the sealing member 56 to prevent foreign substances from attaching to the sealing member 56 when not in use.

The sealing member 56 and the outer layer 58 function in a similar manner to a sticker, wherein the backing of the sticker must be removed to attach the sticky surface to an object. It is appreciated that the opening 29 of the pouch 20 may be sealed utilizing a plurality of various manners rather than the preferred method, such as but not limited to a self-sealing extremity or a thermo-sealing blade.

### D. New Embodiment

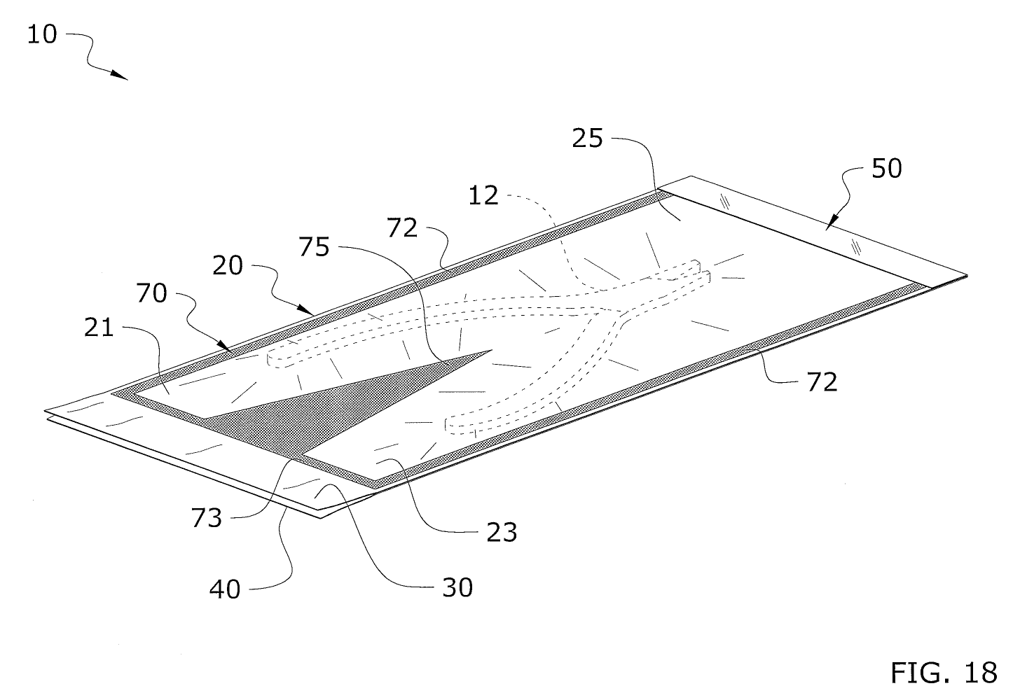

The new embodiment includes the sealing edge 70 that extends around the inner space so as to adequately contain the medical instrument 12 within the pouch 20, wherein the sealing edge 70 sealably connects the first layer 30 to the second layer 40. At least one of the layers 30, 40 is permeable to allow gas or steam to escape, and both of the layers 30, 40 are impermeable with respect to microorganisms entering the layers 30, 40. The sealing edge 70 generally extends along an outer perimeter of the pouch 20 as shown in FIGS. 18 through 26.

The sealing edge 70 includes side portions 72 that extend along the perimeter of the sides of the pouch 20 and an end portion 73 that extends parallel with the perimeter of the end of the pouch 20. The end portion 73 of the sealing edge 70 may be offset, and thus inwardly spaced, from the outer end of the pouch 20 depending on the size of the pouch 20 and the size of the desired medical instrument 12 that is to be enclosed. This also allows for the user a method to open the pouch 20 by peeling apart the first layer 30 from the second layer 40.

An inner portion 75 may also extend inwardly from the end portion 73 of the sealing edge 70 so as to define an enclosed space between the legs of the medical instrument 12. The inner portion 75 may be comprised of various shapes and configurations (e.g. triangular, ovular, rectangular, etc.) as illustrated in FIGS. 18 through 26 all of which at least partially separate the legs of the medical instrument 12 contained within the pouch 20.

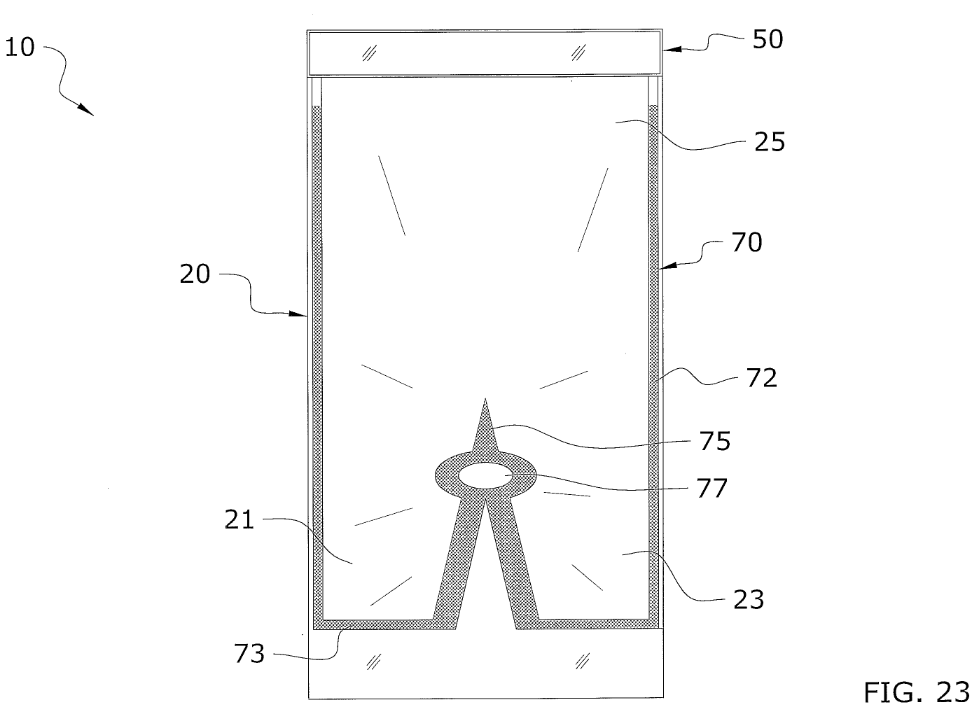

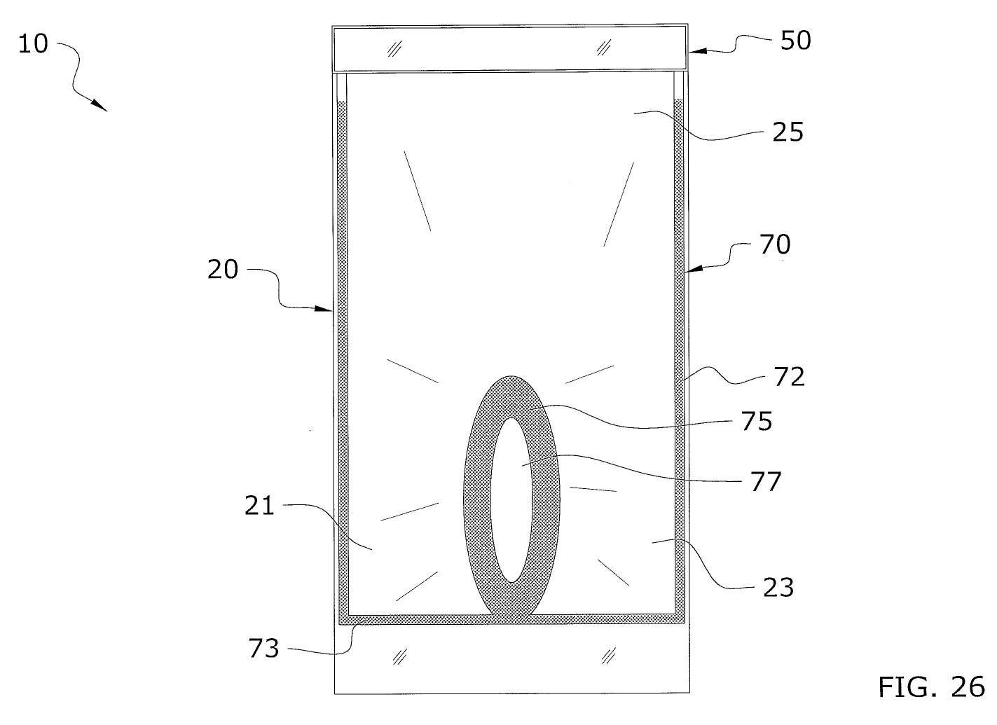

The inner portion 75 allows for the pouch 20 to be manipulated in various ways so as to store the pouch 20 utilizing various hangers, etc. so that the medical instruments 12 will be able to be sterilized adequately within the pouch 20. For example, a cutout 77 (i.e. hole, slit, etc.) may extend through the inner portion 75 as illustrated in FIG. 23. The cutout 77 would allow for the pouch 20 and contained medical instrument 12 to be hung up by extending the hanger through the cutout 77. Another cutout 77 is illustrated in FIG. 26. In these Figures, the cutouts 77 are surrounded by the inner portion 75 of the sealing edge 70 on all sides.

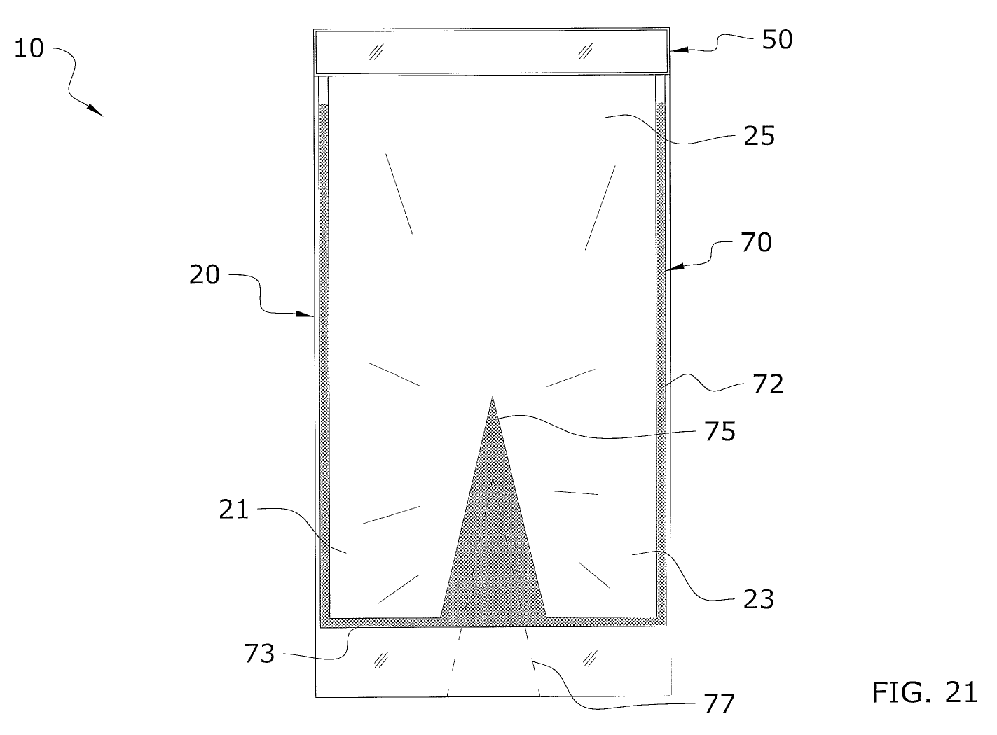

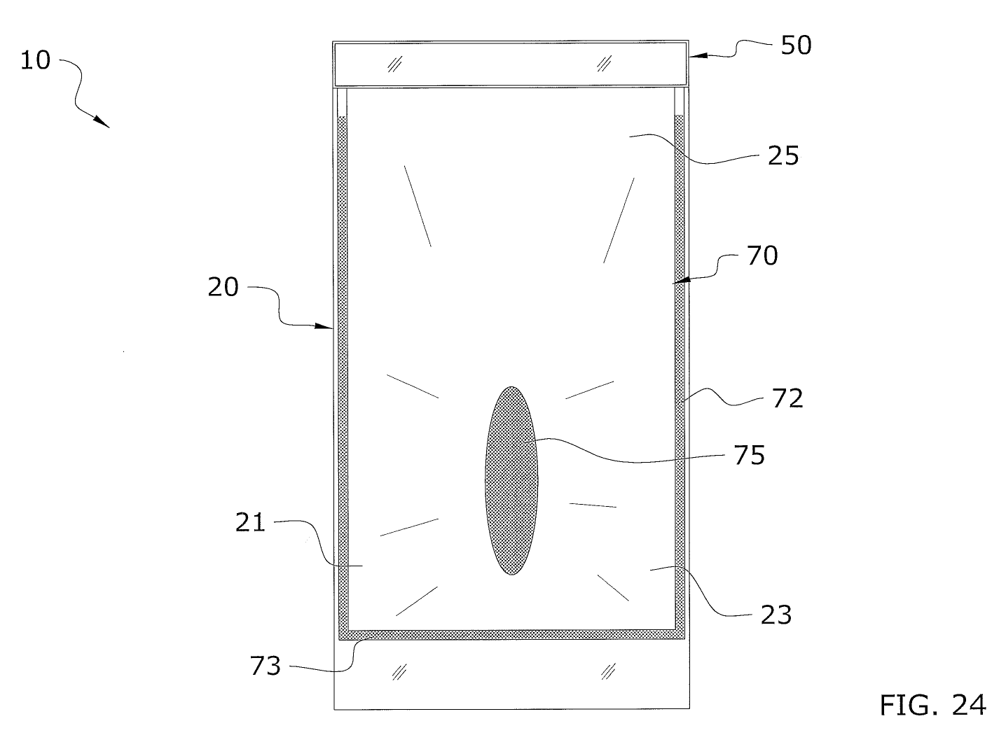

It is appreciated that the cutouts 77 may be preformed as illustrated in FIGS. 23 and 26, or the cutout 77 may be cut within the pouch 20 after purchase as illustrated by the triangular dashed lines extending through the inner portion 75 in FIG. 21. Cutting within the inner portion 75, so as not to extend within the inner space in which the medical instrument 12 is enclosed, allows for various recessed portions or cutouts to be formed to hang or suspend the pouch 20 in various manners. Various other types of cutouts 77 may be formed within the inner portion 75 of the sealing edge 70 as desired with the present invention. It is also appreciated that the inner portion 75 may be left without a cutout 77 in alternate embodiments, wherein the inner portion 75 is completely sealed or enclosed as illustrated in FIGS. 18, 19, 22, 24, and 25. Another configuration, as appreciated in FIG. 24, shows the inner portion 75 separated from the end portion 73 of the sealing edge 70. It is appreciated that various Figures may be combined with each other to form more alternate configurations.

### E. Manufacture of Invention

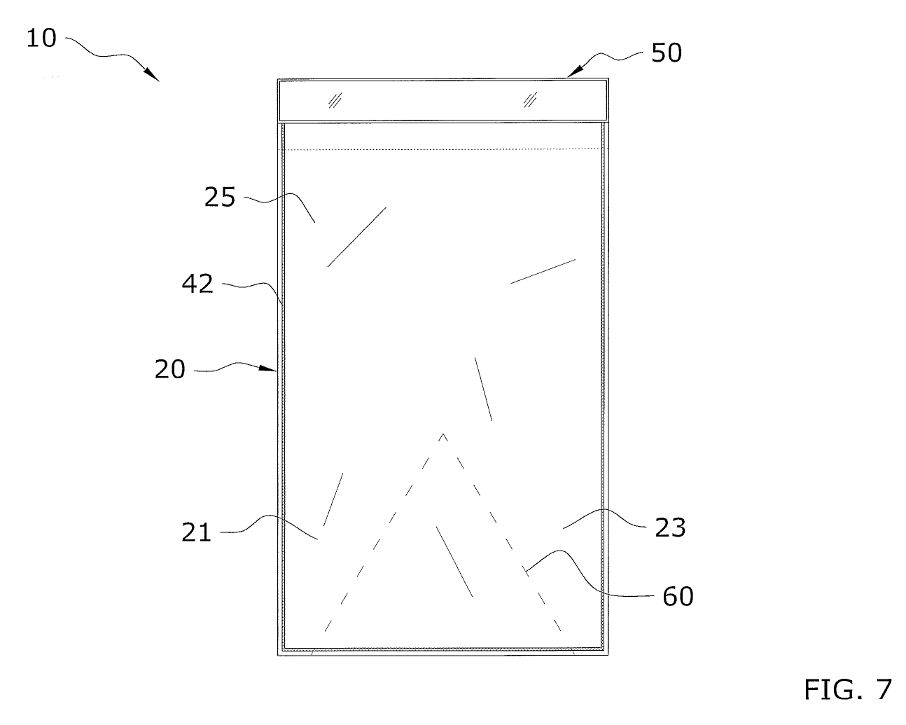

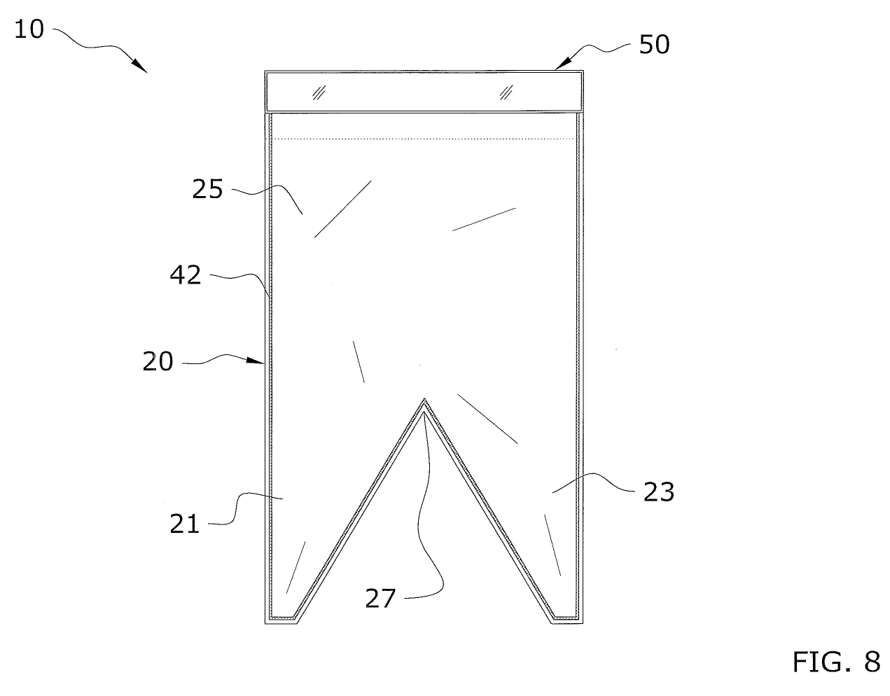

The present invention may be manufactured in various manners as illustrated in FIGS. 7 through 11. A first example of a way to manufacture the present invention involves utilizing a standard rectangular pouch 20 as illustrated in FIGS. 7 and 8. A wedge is cut out of the pouch 20 from a substantially center bottom edge of the pouch 20 as illustrated by the cutting line 60 in FIG. 7. The wedge is preferably comprised of a substantially triangular shaped configuration. The wedge is then removed from the bottom of the pouch 20, thus forming a first lower portion 21, a second lower portion 23 and a recessed portion 27 between thereof. The perimeter of the recessed portion 27 is then sealed to prevent any microorganisms or toxins from entering the inside of the pouch 20 via the recessed portion 27.

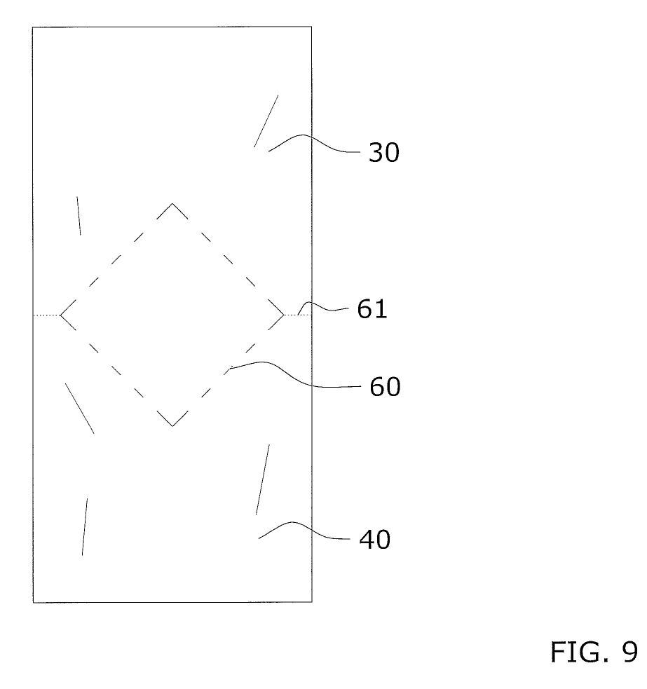

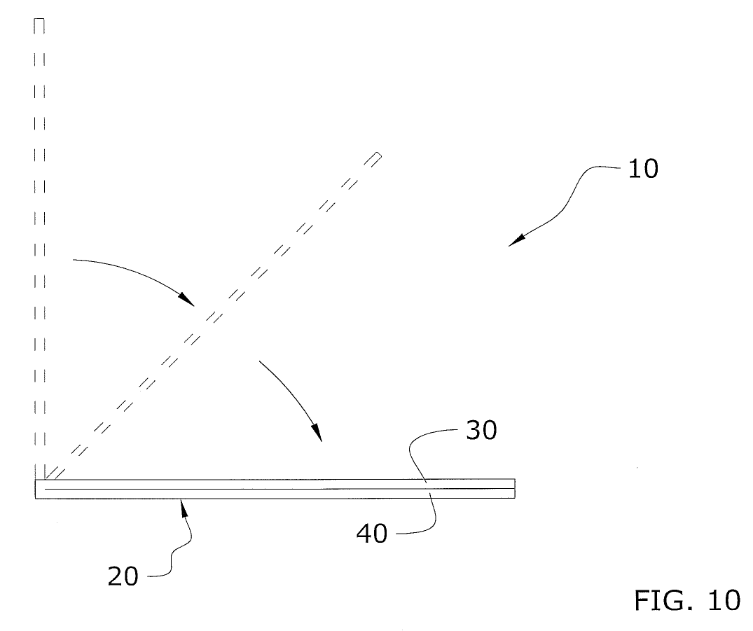

A second example of a way to manufacture the present invention is illustrated in FIGS. 9 and 10. A quadrilateral shaped hole is first cut through a substantial center of the sheet as illustrated by the cutting line 60 in FIG. 9. The sheet is preferably comprised of a pouch 20 material. The quadrilateral shaped hole is preferably cut, wherein each opposing corner perpendicularly faces an outer edge of the sheet as illustrated in FIG. 9. The quadrilateral shaped hole is then removed from the sheet, thus forming a hole in the center of the sheet. The sheet is then folded in the middle as illustrated by the folding line 61 in FIG. 9. The opposing ends of the sheet are thus joined and form a pouch 20 as shown in FIG. 10. The pouch 20 may then be sealed around the outer perimeter of the pouch 20. An opening 29 is also preferably left unsealed to allow insertion of a medical instrument 12. It is appreciated that the present invention may be manufactured in a plurality of manners other than the described manners above.

### F. In Use

In use, the medical instrument 12 (i.e. surgical pliers) is first inserted within the pouch 20 by inserting the handles or legs of the medical instrument 12. Each respective leg is inserted within the respective lower portion 21, 23 of the pouch 20 and the head of the medical instrument 12 is inserted within the upper portion 25 of the pouch 20. When the medical instrument 12 is adequately positioned within the sealed pouch 20 the medical instrument 12 may be sterilized via various techniques, such as but not limited to steam sterilization. It is appreciated that the medical instrument 12 may be sterilized in a plurality of manners and also before insertion into the pouch 20.

The outer layer 58 of the flap 50 is then removed thus revealing the sealing member 56. The second flange portion 54 is then folded over the opening 29 of the pouch 20 and the sealing member 56 is attached to the upper surface of the first layer 30 thus sealing the medical instrument 12 within the pouch 20. The medical instrument 12 may now be positioned upon the respective tool rack 14 (i.e. pliers rack). In the new embodiment, a hanging device may simply be extended through the cutout 77 to suspend or hang the pouch 20. When the user is ready to utilize the medical instrument 12 the above process is simply reversed. The sealed pouch 20 can also be opened by utilizing a scissors to cut open the pouch 20. In addition, a slit 44 anywhere along the non-sealed periphery of the pouch 20, as shown in FIG. 2, would allow the operator to tear open the pouch 20 with their fingers.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention, which is intended to be defined by the following claims (and their equivalents) in which all terms are meant in their broadest reasonable sense unless otherwise indicated. Any headings utilized within the description are for convenience only and have no legal or limiting effect.

I claim:

1. A medical instrument sterilization pouch, comprising:
a first layer comprised of a gas permeable material;
a second layer comprised of a gas impermeable or permeable material; and
a sealing edge substantially extending along an outer perimeter of said first layer and said second layer to attach said first layer to said second layer;
wherein said sealing edge defines a sealed inner space between said first layer and said second layer for receiving a medical instrument;
wherein said sealing edge includes an inner portion extending inwardly from an outer end of said sealing portion within said inner space to define a barrier between a first lower portion and a second lower portion of said pouch.

2. The medical instrument sterilization pouch of claim 1, wherein said inner portion is completely sealed.

3. The medical instrument sterilization pouch of claim 1, wherein said sealing edge includes a pair of side portions extending along said outer perimeter along said sides of said pouch and an end portion inwardly offset from an outer perimeter along said end of said pouch.

4. The medical instrument sterilization pouch of claim 3, wherein said inner portion extends inwardly from said end portion.

5. The medical instrument sterilization pouch of claim 4, wherein said inner portion is separated from said end portion.

6. The medical instrument sterilization pouch of claim 1, including a cutout extending through said inner portion.

7. The medical instrument sterilization pouch of claim 6, wherein said cutout is sealed along an outside perimeter of said cutout via said inner portion.

8. The medical instrument sterilization pouch of claim 6, wherein said cutout is comprised of an ovular shaped configuration.

9. The medical instrument sterilization pouch of claim 6, wherein said cutout is comprised of a triangular shaped configuration.

10. The medical instrument sterilization pouch of claim 1, wherein said inner portion is comprised of a triangular shaped configuration.

11. The medical instrument sterilization pouch of claim 1, wherein said first layer and said second layer are separably formed substantially along at least one outer edge thus forming an opening.

12. The medical instrument sterilization pouch of claim 11, including a flap extending from said pouch, wherein said flap seals said opening.

13. The medical instrument sterilization pouch of claim 1, wherein said first lower portion and said second lower portion each receive a respective leg of a hinged instrument.

14. A medical instrument sterilization pouch, comprising:
a first layer comprised of a gas permeable material;
a second layer comprised of a gas impermeable or permeable material;
a sealing edge substantially extending along an outer perimeter of said first layer and said second layer to attach said first layer to said second layer;
wherein said sealing edge defines a sealed inner space between said first layer and said second layer for receiving a medical instrument;
wherein said sealing edge includes an inner portion extending inwardly from an outer end of said sealing portion within said inner space to define a barrier between a first lower portion and a second lower portion of said pouch; and
a cutout extending through said inner space for hanging said pouch.

15. The medical instrument sterilization pouch of claim 14, wherein said cutout is sealed along an outside edge of said cutout via said inner portion.

16. The medical instrument sterilization pouch of claim 14, wherein said cutout extends through said outer perimeter of said pouch.

17. The medical instrument sterilization pouch of claim 14, wherein said cutout is comprised of an ovular shaped configuration.

18. The medical instrument sterilization pouch of claim 14, wherein said cutout is comprised of a triangular shaped configuration.

19. The medical instrument sterilization pouch of claim 14, wherein said first lower portion and said second lower portion each receive a respective leg of a hinged instrument.

20. A medical instrument sterilization pouch, comprising:
a first layer comprised of a gas permeable material;
a second layer comprised of a gas impermeable or permeable material;
a sealing edge substantially extending along an outer perimeter of said first layer and said second layer to attach said first layer to said second layer;
wherein said sealing edge defines a sealed inner space between said first layer and said second layer for receiving a medical instrument;
wherein said sealing edge includes an inner portion extending inwardly from an outer end of said sealing portion within said inner space to define a barrier between a first lower portion and a second lower portion of said pouch;
wherein said sealing edge includes a pair of side portions extending along said outer perimeter along said sides of said pouch and an end portion inwardly offset from an outer perimeter along said end of said pouch;
wherein said inner portion extends inwardly from said end portion;
a cutout extending through said inner portion;
wherein said first layer and said second layer are separably formed substantially along at least one outer edge thus forming an opening; and
a flap extending from said pouch, wherein said flap seals said opening;
wherein said first lower portion and said second lower portion each receive a respective leg of a hinged instrument.
